(19) United States
(12) Patent Application Publication
(10) Pub. No.: US 2011/0112202 A1
(43) Pub. Date: May 12, 2011

(54) HYDRODENITROGENATION OF HYDROCARBON COMPOUNDS CONTAINING NITRILE OR AMINE FUNCTIONS

(75) Inventors: Philippe Marion, Vernaison (FR); Amélie Hynaux, Plaisir (FR); Dorothée Laurenti, Rillieux-la-pape (FR); Christophe Geantet, Miribel (FR)

(73) Assignees: RHODIA OPERATIONS, AUBERVILLIERS (FR); CENTRE NATIONAL DE LA RECHERCHE SCIENTIFIQUE, PARIS (FR)

(21) Appl. No.: 12/812,904

(22) PCT Filed: Jan. 9, 2009
(86) PCT No.: PCT/EP2009/050194

§ 371 Date: Jan. 21, 2011

(30) Foreign Application Priority Data
Jan. 18, 2008 (FR) 0800256

(51) Int. Cl. C07C 27/00; C07C 1/32; C01C 1/08

(52) U.S. Cl. 518/702; 585/357; 585/733; 423/358

(57) ABSTRACT

Hydrocarbon compounds containing at least one nitrile or amine functional group, e.g., methylglutaronitrile or ortho-toluenediamine, are converted, via hydrodenitrogenation, into ammonia, hydrogen, carbon monoxide and hydrocarbon compounds, notably into hydrocarbon compounds having a low number of carbon atoms, such as methane, or into ammonia.

The present invention relates to a process for treating hydrocarbon compounds containing at least one nitrile or amine function.

It relates more particularly to a process of treatment that involves converting hydrocarbon compounds containing at least one nitrile or amine function to ammonia, hydrogen, carbon monoxide and hydrocarbon compounds, especially to hydrocarbon compounds containing a low carbon number.

Numerous industrial processes generate effluents which comprise hydrocarbon compounds containing nitrile or amine functions. Such effluents cannot be discharged to the environment without treatment. When the concentration of these compounds in the effluents generated is low, a number of treatment processes have been proposed, such as incineration, biological treatments, nitrification or adsorption processes. When, however, the concentration of compounds containing amine or nitrile functions is high, or these nitrile or amine compounds are by-products which cannot be directly exploited in an industrial process for preparing chemical products, it is preferable for the economics of these processes and for the environment to recycle these compounds without conversion or after conversion to products which are directly utilizable in the process or even in other processes.

An example of an industrial process generating effluents containing a high concentration of compounds containing at least one nitrile function, or nitrile by-products, is the process for preparing adiponitrile by hydrocyanation of butadiene, which has been exploited industrially since 1970.

Thus the compound 2-pentenenitrile (2-PN) does not react with hydrocyanic acid to form a dinitrile, and is recovered by distillative separation in the form of a stream of unexploitable by-products. Similarly, the 2-methylglutaronitrile (MGN) formed in the second hydrocyanation step cannot be exploited for hexamethylene diamine. These unexploitable by-products are usually destroyed by incineration in boilers for producing steam.

However, some of these by-products may be exploited completely or partially by chemical conversion to new, useful compounds. Thus the major by-product in terms of quantity in the preparation of adiponitrile, 2-methylglutaronitrile (MGN), may especially be hydrogenated to produce a branched diamine, 2-methylpentamethylenediamine (MPMD), which is used principally as a monomer for the preparation of polyamide or as a starting material for the synthesis of chemical products. Other exploitations of MGN have been described.

The other dinitrile or mononitrile by-products are essentially exploited by combustion to produce energy. Since, however, these compounds contain nitrogen atoms, the combustion gases contain oxides of nitrogen. It may therefore be necessary to treat the combustion gases in units for converting and destroying nitrogen oxides that are referred to as DENOx units.

The industrial processes for synthesis of 2,4- and 2,6-toluenediamines (TDA) give rise to by-products which must be destroyed on account of their low economic interest, namely the mixture of isomers of the ortho-toluenediamines.

The problem of treating and exploiting unexploitable by-products in—especially—the process of hydrocyanating butadiene and the process of preparing toluenediamine has therefore still not been entirely solved, and new solutions are continually being sought.

One of the aims of the present invention is to provide a process for treating these compounds that does not have the drawbacks of combustion or incineration and that allows the overall economics of the process to be improved, especially by converting said compounds into the form of compounds which are exploitable and, advantageously, recyclable.

The invention accordingly provides a process for treating hydrocarbon compounds containing at least one nitrile or amine function by conversion to exploitable compounds, characterized in that it comprises treating said compounds in a hydrodenitrogenation or hydrotreating step by reaction with hydrogen under an absolute pressure of between 0.1 and 10 MPa, preferably from 0.5 MPa to 3 MPa, at a temperature of between 200° C. and 500° C., preferably from 300° C. to 400° C., in the presence of a hydrodenitrogenation catalyst in order to convert these compounds to ammonia and hydrocarbon compounds.

The process of the invention therefore allows, for example, the treatment of some or all of the stream of unexploitable compounds containing nitrile or amine functions that are generated in the processes of hydrocyanating olefins, more particularly butadiene, or in the processes for preparing toluenediamine, in order to recover the nitrogen atom in ammonia form and the majority of the carbon and hydrogen atoms in the form of hydrocarbon compounds containing 1 to a plurality of carbon atoms. These hydrocarbon compounds may be exploited as they are or fed to a steam reforming and, optionally, methanation step, in order to be converted either to carbon monoxide and hydrogen or to methane, these products being exploitable in particular as a generator of energy, but also as a starting material for the synthesis of numerous compounds. Accordingly, and as an example, hydrogen may be used in numerous chemical compound production processes, such as the hydrogenation of adiponitrile or of dinitrotoluene; carbon monoxide may be used in the process for synthesis of phosgene; and methane may be used in the synthesis of hydrocyanic acid.

According to another feature of the invention, the hydrodenitrogenation catalyst comprises a metallic element belonging to the group of noble metals consisting of platinum, palladium, rhodium, ruthenium or to the transition elements such as nickel.

Advantageously and preferably the catalyst is of the supported catalyst type, in which the metallic catalytic element is supported on a material, preferably a porous material, such as alumina, silica, aluminosilicates, silica-aluminas, activated carbons, zirconia, titanium oxide and zeolites.

The preferred catalyst of the invention comprises platinum deposited on a support selected from the group consisting of silica, zirconia, aluminosilicates, silica-aluminas and zeolites.

The hydrodenitrogenation reaction is carried out in the presence of a heterogeneous catalyst which is either dispersed in suspension in the reactor or is in the form of a fixed bed or fluidized bed through which the stream of nitrile or amine compounds is fed. The catalyst may also be deposited on a monolithic support such as, for example, a honeycomb-form support.

The present invention is not limited to these embodiments, which are given solely as an illustration.

The preferred hydrodenitrogenation catalysts of the invention are, in particular, platinum-on-zirconia, platinum-on-aluminosilicate, platinum-on-silica-alumina and platinum-on-zeolite catalysts.

The degree of conversion of the compounds to be treated that are employed is very high, close to or equal to 100%. The products recovered are ammonia and, for the major part, hydrocarbon compounds. For instance, the treatment of 2-methylglutaronitrile produces, as hydrocarbon compounds, 2-methylpentane very much in the majority. The hydrodenitrogenation of ortho-toluenediamine leads primarily to the production of methylcyclohexane. The ammonia is separated off and recovered, especially by distillation.

This hydrotreating may also be accompanied by thermal cracking of the hydrocarbon chains, leading to the formation of hydrocarbon compounds without a nitrogen atom and/or of hydrocarbon compounds containing nitrogen atoms. The latter can be converted to hydrocarbon compounds by reaction with hydrogen, according to the operating conditions employed. Furthermore, cyclic compounds containing nitrogen atoms may also be formed, such as picoline or its derivatives and piperidines, in the case of the hydrotreating of MGN. According to the invention, the term % HDN is applied to the ratio expressed as a percentage of the number of moles of hydrocarbon compounds containing no nitrogen atoms that are produced either by hydrotreating or by thermal cracking, relative to the number of moles of compounds to be treated that are employed.

According to one preferred characteristic of the invention, the hydrocarbon compounds produced by hydrodenitrogenation or hydrotreating, such as 2-methylpentane, and products of thermal cracking may be subjected to steam reforming, allowing partial oxidation of these compounds to carbon monoxide (CO) and hydrogen (H₂). These two products may be recovered and exploited directly as a mixture or after purification and separation. In this embodiment it is preferable to remove the traces of ammonia present in the hydrocarbon compounds, so as not to detract from the efficiency of the steam reforming.

According to another embodiment of the invention this mixture of carbon monoxide and hydrogen may be subjected to a methanation reaction, leading to the formation of water and alkanes with a low carbon number such as methane. This steam reforming/methanation treatment is widely used in the petroleum industry. Typical catalysts for these reactions include supported nickel catalysts. The implementation temperature is between 400 and 700° C. for steam reforming and between 200 and 400° C. for methanation.

A general description of the processes of steam reforming and methanation is given in the work “Les procêdês de pêtrochimie”, TECHNIP, Volume 1, 1965, its authors being A. CHAUVEL, G. LEFEBVRE and L. CASTEX.

The process of the invention is applied in particular to the process for preparing adiponitrile by hydrocyanation of butadiene in two steps. This process is described in numerous patents, and a detailed description is available in RAPPORTS SRI 31, suppl. B, entitled “HEXAMETHYLENEDIAMINE”.

It also applies to the process for preparing toluenediamine that is described in numerous documents and especially in Rapports SRI 1, supplement B “Isocyanates”.

Other advantages and details of the invention will emerge more clearly from the examples given below solely by way of illustration.

The tests described below were carried out with two hydrodenitrogenation catalysts:

Catalyst A: Pt deposited on zirconia (Pt/ZrO₂)

Catalyst B: Platinum deposited on a silica-alumina support comprising a weight percentage of silica of 10, referred to as Pt/SiAl10.

Catalyst A was obtained using a zirconia support with a specific surface area of 83 m²/g.

Catalyst B comprises a silica-alumina support with a specific surface area of 352 m²/g which is sold by Condêa under the trade name SIRAL 10. This support contains 10% by weight of SiO₂.

These catalysts are prepared by the procedure below.

The supports are impregnated with a solution of hexachloroplatinic acid H₂PtCl₆. They are left to age at ambient temperature for two hours to allow the solution to penetrate the pores. The products are then dried overnight (>12 h) at 110° C. and subsequently calcined in a stream of air at 500° C. for 1 hour (air flow rate of 60 cm³·min⁻¹, temperature rise ramp of 2° C.·min⁻¹), in order to decompose the precursor complex to form platinum oxide. They are then reduced in a stream of hydrogen at 310° C. for 6 hours (hydrogen flow rate of 60 cm³·min⁻¹, temperature rise ramp of 1° C.min⁻¹) to give a deposit of metallic platinum.

The physicochemical characteristics of the Pt/ZrO₂ and Pt/SiAl10 catalysts are collated in Table I.

The dispersion and the platinum particle size were determined by hydrogen chemisorption. The platinum was assayed by a plasma emission spectrometry.

TABLE I

| | % by mass of | dispersion | s_{particle} |
|---|---|---|---|
| Catalyst | Pt | [%] | [nm] |
| A | 1.1 | 60 | 1.7 |
| B | 1.1 | 66 | 1.4 |

In the examples which follow, the abbreviations used have the meanings indicated below:
MP: 2-methylpentane
Pic: picolines (β-picoline, 2-amino-3-picoline, 6-amino-3-picoline)
% HDN: percentage of hydrocarbon products containing no hydrogen atoms, relative to the number of moles of compounds to be treated.

### EXAMPLE 1

### Hydrodenitrogenation of MGN under an Absolute pressure of 0.1 MPa using catalyst A.

The hydrodenitrogenation (HDN) reaction of methylglutaronitrile was carried out at different temperatures and under an absolute pressure of 0.1 MPa with a hydrogen flow rate of 55 ml/min and a fixed bed of catalyst A with a mass of 15 mg, in accordance with the procedure below, in a dynamic microreactor.

The reaction mixture comprises pure 2-methyl-glutaronitrile and hydrogen. The hydrogen, whose flow rate is regulated by a mass flow meter (0-200 ml/min), bubbles into a saturator which is filled with liquid MGN, and then passes into a condenser, whose temperature controls the partial pressure of MGN to give an MGN partial pressure of 1.33 kPa. The reactor is placed in a tubular oven whose temperature is controlled by a platinum probe regulator. The reaction temperature is measured by a thermocouple situated in the catalyst bed.

In order to prevent condensation of the reactant and of the reaction products, the temperature of the apparatus assembly is consistently maintained at 180° C. A trap is sited at the exit of the test in order to condense the reaction products and the unconverted reactant. The gases then exit via the vent.

The concentration and the number of moles of each compound present in the condensed medium are determined by gas-chromatographic analysis. The different yields obtained are collated in Table II below:

TABLE II

| T [° C.] | 250 | 300 | 350 | 400 | 450 |
|---|---|---|---|---|---|
| Nitrogen-containing | 70.3 | 78.6 | 74 | 64.9 | 67 |
| products (including | (3.6) | (65) | (57.6) | (27.7) | (10.9) |
| Pic) [%] | | | | | |
| Hydrocarbon products | 0.3 | 2.6 | 3.7 | 13 | 12 |
| (including MP) [%] | (0.3) | (1.7) | (1.2) | (0.7) | (0.2) |

### EXAMPLE 2

### Hydrodenitrogenation of MGN under an Absolute Pressure of 0.1 MPa (MGN Partial Pressure=1.33 kPa) Using Catalyst B.

Example 1 is repeated with the exception of the type of catalyst, which is catalyst B.

The yields obtained are collated in Table III below:

TABLE III

| T [° C.] | 250 | 300 | 350 | 400 | 450 |
|---|---|---|---|---|---|
| Nitrogen-containing | 61.3 | 68.3 | 65.7 | 58.7 | 43.8 |
| products (including | (4.4) | (57.5) | (48) | (25.9) | (9.9) |
| Pic) [%] | | | | | |
| Hydrocarbon products | 0.3 | 1.4 | 4.8 | 18.3 | 40.4 |
| (including MP) [%] | (0.3) | (1.1) | (1.4) | (1.2) | (0.7) |

### EXAMPLE 3

### Hydrodenitrogenation of MGN at an Absolute Pressure of 0.55 MPa (MGN Partial Pressure=1.33 kPa) Over Catalyst B.

Example 1 is repeated, using 50 mg of catalyst A under an absolute pressure of 0.55 MPa and a hydrogen flow rate of 4 ml/min. When the tests are carried out under pressure, the reaction mixture is injected after letdown to atmospheric pressure in a gas chromatograph via a six-way valve.

The yields obtained are collated in Table IV below:

TABLE IV

| T [° C.] | 250 | 300 | 350 |
|---|---|---|---|
| Nitrogen-containing | 30.2 | 0 | 0.3 |
| products (including | (2.5) | | (0.3) |
| Pic) [%] | | | |
| Hydrocarbon products | 69.8 | 100 | 99.7 |
| (including MP) [%] | (68.6) | (93.9) | (78.5) |

### EXAMPLE 4

### Hydrodenitrogenation of MGN Under an Absolute Pressure of 1 MPa and an MGN Partial Pressure Of 1.33 kPa Using Catalyst B.

Example 1 is repeated with the exception of the type of catalyst, which is catalyst B.

The yields obtained are collated in Table V below:

TABLE V

| T [° C.] | 250 | 300 | 350 | 400 |
|---|---|---|---|---|
| Nitrogen-containing | 65.6 | 4.4 | 0 | 3.4 |
| products (including | (0.9) | (1.3) | | (3.4) |
| Pic) [%] | | | | |
| Hydrocarbon products | 34.4 | 95.6 | 100 | 96.6 |
| (including MP) [%] | (32.9) | (90.7) | (86.3) | (54.9) |

These results show that the conversion of MGN to hydrocarbon compounds is low under a pressure of 0.1 MPa for a temperature of between 250° C.<T<350° C., which demonstrates low activity of the catalyst in these operating conditions.

Under a pressure of 1 MPa, the yield from the conversion of MGN to hydrocarbon compounds is higher, and reaches a value of 100% for a temperature of 350° C.

Under a pressure of 0.55 MPa it is also possible to obtain a yield of 100% for this conversion of MGN to hydrocarbon compounds, for a temperature of 300°.

### EXAMPLE 5

The hydrodenitrogenation reaction of ortho-toluenediamine (OTD) was carried out under an absolute pressure of 1 MPa in a device identical with that of Example 1, with a hydrogen flow rate of 20 ml/min and a mass of catalyst A of 50 mg.

The reaction mixture is composed of hydrogen and a mixture obtained as a by-product in a plant for producing toluenediamine (TDA), comprising essentially 2,3-diaminotoluene and 3,4-diaminotoluene. The hydrogen, whose flow rate is regulated by a mass flow meter (0-200 ml/min), bubbles into a saturator which is filled with melted OTD, and then passes into a condenser whose temperature controls the partial pressure of OTD. In the example under consideration, the absolute pressure is 1 MPa, with an OTD partial pressure of 1.33 kPa, the conditioning temperature being 140° C.

The reactor used under a pressure of 1 MPa is made of stainless steel (internal diameter 10 mm, length 40 mm). It is placed in a tubular oven whose temperature is controlled by a platinum probe regulator. The reaction temperature is measured by a thermocouple which is situated in the catalyst bed.

When the catalytic tests are carried out under pressure (1 MPa), a capillary is sited at the outlet of the reactor. It allows an upstream pressure to be maintained in the apparatus that is a function of the flow rate used and of the length and diameter of the capillary. Following letdown to atmospheric pressure, the reaction mixture is injected into a gas chromatograph via a six-way valve.

To prevent the condensation of the reactant and of the reaction products, the temperature of the apparatus assembly is consistently heated at 180° C. A trap is sited at the outlet from the test to condense the reaction products and the unconverted reactant. The gases subsequently exit at the vent.

Analysis of the reaction mixture is completely automated and is carried out online by gas chromatography (Hewlett Packard chromatograph equipped with a flame ionization detector, an HP 3396 series II integrator and a DB1 capillary column with dimensions of 50 m×0.32 mm×5 μm).

| T [° C.] | 300 | 350 |
|---|---|---|
| OTD [%] | 0 | 0 |
| % HDN | 98 | 100 |

The great majority of the methylcyclohexane is obtained at 300° C. At 350° C., significant presence of toluene and of methylcyclohexane is recorded.

### EXAMPLE 6

### Steam Reforming of the Hydrocarbon Compounds Produced, Such as Methylpentane

A stream of 5 g/h of methylpentane is fed to a reactor in gas phase in parallel with a stream of water of 7.5 g/h. The reactor contains approximately 100 ml of a nickel-based catalyst supported on alumina (70% of nickel). The temperature is maintained at about 550° C. by external heating. The pressure is regulated at 23 bar. On exiting, the gas is cooled and then analysed. The conversion of the methylpentane is complete. Only CO, hydrogen and, to a lesser extent, CO₂ are detected.

1.-10. (canceled)

11. A process for the conversion of at least one hydrocarbon compound containing at least one nitrile or amine functional group into ammonia and at least one hydrocarbon compound, comprising hydrodenitrogenating said at least one hydrocarbon compound containing at least one nitrile or amine functional group with hydrogen under an absolute hydrogen pressure ranging from 0.1 to 10 MPa at a temperature ranging from 200° C. to 500° C. and in the presence of a hydrodenitrogenation catalyst.

12. The process as defined by claim 11, wherein the hydrodenitrogenation catalyst comprises a metallic element selected from the group consisting of platinum, palladium, rhodium, ruthenium and nickel.

13. The process as defined by claim 12, wherein the hydrodenitrogenation catalyst comprises a metallic element supported on a support selected from the group consisting of alumina, silica, aluminosilicates, silica-aluminas, activated carbons, zirconia and titanium oxide.

14. The process as defined by claim 13, wherein the hydrodenitrogenation catalyst comprises platinum deposited on a support selected from the group consisting of zirconia, silica, alumina, aluminosilicate and silica-alumina.

15. The process as defined by claim 11, wherein the absolute hydrogen pressure ranges from 0.5 MPa to 3 MPa.

16. The process as defined by claim 11, carried out at a temperature ranging from 300° C. to 400° C.

17. The process as defined by claim 11, comprising the hydrodenitrogenation of at least one nitrile compound selected from the group consisting of methylglutaronitrile, ethylsuccinonitrile, 2-pentenenitrile, 2-methyl-2-butenenitrile or mixtures thereof and the isomers of ortho-TDA.

18. The process as defined by claim 11, comprising treating at least one hydrocarbon compound recovered upon completion of the hydrodenitrogenation in a steam reforming step and producing carbon monoxide and hydrogen.

19. The process as defined by claim 18, further comprising treating said carbon monoxide and the hydrogen via methanation and producing at least one lower alkane.

20. The process as defined by claim 19, wherein the step of steam reforming and methanation is carried out in the presence of a supported nickel-based catalyst at a temperature ranging from 400° C. to 700° C. for the steam reforming and from 200° C. to 400° C. for the methanation.
